(19) United States
(12) Patent Application Publication
(10) Pub. No.: US 2024/0000439 A1
(43) Pub. Date: Jan. 4, 2024

(54) ULTRASOUND DIAGNOSTIC APPARATUS AND CONTROL METHOD FOR ULTRASOUND DIAGNOSTIC APPARATUS

(71) Applicant: FUJIFILM Corporation, Tokyo (JP)

(72) Inventor: Yukiya MIYACHI, Kanagawa (JP)

(73) Assignee: FUJIFILM Corporation, Tokyo (JP)

(21) Appl. No.: 18/468,339

(22) Filed: Sep. 15, 2023

Related U.S. Application Data
(63) Continuation of application No. PCT/JP2022/009978, filed on Mar. 8, 2022.

(30) Foreign Application Priority Data
Mar. 26, 2021 (JP) 2021-052639

(51) Int. Cl. A61B 8/00

(52) U.S. Cl. CPC A61B 8/54; A61B 8/4245; A61B 8/461

(57) ABSTRACT

According to the present invention, in an ultrasound diagnostic apparatus and a control method for an ultrasound diagnostic apparatus, an ultrasound image generation unit is configured to generate an ultrasound image from a reception signal obtained by transmitting and receiving an ultrasound beam to and from an examination area of a subject under examination using the ultrasound probe, and an optical camera is configured to generate an optical image by imaging the subject under examination including the ultrasound probe during examination of the examination area. A probe recognition section is configured to recognize the ultrasound probe from the optical image, and a trimming section is configured to generate a trimmed image by trimming a region including the ultrasound probe from the optical image in a case where the ultrasound probe is recognized. Then, a display control unit is configured to display the ultrasound image and the trimmed image on a monitor. With this, it is possible to enlarge and display an optical image representing a position and an orientation of the ultrasound probe during the examination of the examination area on the monitor.

### CROSS-REFERENCE TO RELATED APPLICATIONS

This application is a Continuation of PCT International Application No. PCT/JP2022/009978 filed on Mar. 8, 2022, which claims priority under 35 U.S.C. § 119(a) to Japanese Patent Application No. 2021-052639 filed on Mar. 26, 2021. The above applications are hereby expressly incorporated by reference, in their entirety, into the present application.

### BACKGROUND OF THE INVENTION

### 1. Field of the Invention

The present invention relates to an ultrasound diagnostic apparatus and a control method for an ultrasound diagnostic apparatus for generating an ultrasound image including an examination area of a subject under examination.

### 2. Description of the Related Art

In recent years, in a case where an examination area of a subject under examination is examined using an ultrasound diagnostic apparatus inside an intensive care unit (ICU) or the like, for example, a first person examines the examination area of the subject under examination using an ultrasound probe, an ultrasound image of the examination area is displayed on a monitor disposed outside the ICU, a second person performs remote diagnosis while viewing the ultrasound image of the examination area displayed on the monitor, in many cases.

However, the second person outside the ICU, who views only the ultrasound image, cannot specify at which position and in which orientation on the subject under examination the ultrasound image is an ultrasound image generated by performing scanning. Therefore, in order to support remote diagnosis, there is an increasing need to display an ultrasound image and an optical image including the ultrasound probe on the monitor at the same time, or to store these images in association with each other.

For example, WO2019-064706A and JP2019-521745A disclose that an ultrasound image and an optical image including an ultrasound probe, which is captured using an optical camera provided in an apparatus main body of a handheld ultrasound diagnostic apparatus are displayed on a monitor of the apparatus main body. Further, JP2009-207800A discloses that an optical camera is attached to a stationary ultrasound diagnostic apparatus, an optical image in which a diagnostic site is zoomed in is captured using this optical camera, and this optical image is recorded together with an ultrasound image.

### SUMMARY OF THE INVENTION

In a case where the examination area of the subject under examination is examined in a handheld ultrasound diagnostic apparatus as in WO2019-064706A and JP2019-521745A, unlike a stationary ultrasound diagnostic apparatus as in JP2009-207800A, a user, for example, holds the apparatus main body in one hand to image the subject under examination including the ultrasound probe during the examination of the examination area by using the optical camera provided in the apparatus main body while holding the ultrasound probe in the other hand to scan the examination area.

Therefore, as in JP2009-207800A, operating the optical camera and zooming in to image the examination area may be quite difficult for the user who is capturing the ultrasound image. For example, in a case where the ultrasound probe is shown at an end part of the optical image, the ultrasound probe may fall outside an angle of view because zooming in typically enlarges the center of the optical image as the center. In addition, in a case where zooming in is performed, the angle of view may become narrower, resulting in frequent changes in the imaging scene.

Therefore, an object of the present invention is to provide an ultrasound diagnostic apparatus and a control method for an ultrasound diagnostic apparatus capable of enlarging and displaying an optical image representing a position and an orientation of an ultrasound probe during examination of an examination area on a monitor.

In order to achieve the above-described object, according to the present invention, there is provided an ultrasound diagnostic apparatus comprising:
an ultrasound probe; and
an apparatus main body connected to the ultrasound probe,
in which the apparatus main body includes
   an ultrasound image generation unit configured to generate an ultrasound image from a reception signal obtained by transmitting and receiving an ultrasound beam to and from an examination area of a subject under examination using the ultrasound probe,
   at least one optical camera configured to generate an optical image by imaging the subject under examination including the ultrasound probe during examination of the examination area,
   a probe recognition section configured to recognize the ultrasound probe from the optical image,
   a trimming section configured to generate a trimmed image by trimming a region including the ultrasound probe from the optical image in a case where the ultrasound probe is recognized,
   a monitor, and
   a display control unit configured to display the ultrasound image and the trimmed image on the monitor.

Here, it is preferable that the at least one optical camera includes a plurality of optical cameras having different angles of view from each other,
that the apparatus main body includes an optical camera selection section configured to select one or more optical cameras from among the plurality of optical cameras based on an instruction from a user, and
that the plurality of optical cameras are disposed on the same-side surface of the apparatus main body.

In addition, it is preferable that the optical camera selection section is configured to select one optical camera from among the plurality of optical cameras based on an instruction from the user,
that the probe recognition section is configured to recognize the ultrasound probe from an optical image generated by the one optical camera, and
that the trimming section is configured to generate the trimmed image from the optical image generated by the one optical camera.

Further, it is preferable that the optical camera selection section is configured to select a first optical camera and a second optical camera from among the plurality of optical cameras based on an instruction from the user,
that the first optical camera is a standard camera that generates a standard optical image by imaging the subject under examination with a first angle of view,
that the second optical camera is a wide-angle camera that generates a wide-angle optical image by imaging the subject under examination with a second angle of view having a wider angle of view than that of the standard camera, and
that the probe recognition section is configured to recognize the ultrasound probe from each of the standard optical image and the wide-angle optical image.

Further, it is preferable that the trimming section is configured to generate the trimmed image from the standard optical image in a case where the ultrasound probe is recognized from both the standard optical image and the wide-angle optical image, and to generate the trimmed image from the wide-angle optical image in a case where the ultrasound probe is recognized from the wide-angle optical image and the ultrasound probe is not recognized from the standard optical image.

Further, it is preferable that the probe recognition section is configured to recognize the ultrasound probe from the wide-angle optical image, and to recognize the ultrasound probe from the standard optical image in a case where the ultrasound probe is recognized from the wide-angle optical image.

Further, it is preferable that the apparatus main body includes a region setting memory configured to store, in the optical image divided into a plurality of regions set in advance, one region set in advance from among the plurality of regions,
that the probe recognition section is configured to recognize the ultrasound probe from only the one region, and
that the trimming section is configured to generate the trimmed image from only the one region.

Further, it is preferable that the probe recognition section is configured to recognize, in the optical image, the ultrasound probe from only a trimmed region set based on an instruction from a user, and
that the trimming section is configured to generate the trimmed image from only the trimmed region.

Further, it is preferable that the probe recognition section is configured to recognize the ultrasound probe in a state of being held by a user's hand from the optical image.

Further, it is preferable that the apparatus main body includes a notification section configured to issue a notification that the ultrasound probe is not recognized in a case where the ultrasound probe is not recognized.

Further, it is preferable that the display control unit is configured to display the optical image on the monitor in a case where the ultrasound probe is not recognized, and to display the trimmed image on the monitor in a case where the ultrasound probe is recognized.

In addition, according to the present invention, there is provided an ultrasound diagnostic apparatus comprising:
an ultrasound probe; and
an apparatus main body connected to the ultrasound probe,
in which the apparatus main body includes
   an ultrasound image generation unit configured to generate an ultrasound image from a reception signal obtained by transmitting and receiving an ultrasound beam to and from an examination area of a subject under examination using the ultrasound probe,
   at least one optical camera configured to generate an optical image by imaging the subject under examination including the ultrasound probe during examination of the examination area,
   a region setting memory configured to store, in the optical image divided into a plurality of regions set in advance, one region set in advance from among the plurality of regions,
   a trimming section configured to generate a trimmed image by trimming the one region from the optical image,
   a monitor, and
   a display control unit configured to display the ultrasound image and the trimmed image on the monitor.

Here, it is preferable that the at least one optical camera includes a plurality of optical cameras having different angles of view from each other,
that the apparatus main body includes an optical camera selection section configured to select one or more optical cameras from among the plurality of optical cameras based on an instruction from a user,
that the plurality of optical cameras are disposed on the same-side surface of the apparatus main body,
that the optical camera selection section is configured to select one optical camera from among the plurality of optical cameras based on an instruction from the user, and
that the trimming section is configured to generate the trimmed image from an optical image generated by the one optical camera.

In addition, it is preferable that the at least one optical camera includes a third optical camera disposed on a surface of the apparatus main body on the same side as the monitor, and
that the apparatus main body includes
   an eye gaze recognition section configured to recognize a user's eye gaze based on an optical image generated by the third optical camera, and
   an apparatus control unit configured to freeze at least one of the ultrasound image or the optical image in a case where an eye gaze behavior set in advance is recognized.

Further, it is preferable that the trimming section is configured to generate the trimmed image by trimming from the optical image, a region including, in addition to the ultrasound probe, the subject under examination in a range representing a position and an orientation of the ultrasound probe during the examination of the examination area.

In addition, according to the present invention, there is provided a control method for an ultrasound diagnostic apparatus, comprising:
a step of generating, via an ultrasound image generation unit, an ultrasound image from a reception signal obtained by transmitting and receiving an ultrasound beam to and from an examination area of a subject under examination using the ultrasound probe;
a step of generating, via at least one optical camera, an optical image by imaging the subject under examination including the ultrasound probe during examination of the examination area;
a step of recognizing, via a probe recognition section, the ultrasound probe from the optical image;
a step of generating, via a trimming section, a trimmed image by trimming a region including the ultrasound probe from the optical image in a case where the ultrasound probe is recognized; and
a step of displaying, via a display control unit, the ultrasound image and the trimmed image on a monitor.

In addition, according to the present invention, there is provided a control method for an ultrasound diagnostic apparatus, comprising:
a step of generating, via an ultrasound image generation unit, an ultrasound image from a reception signal obtained by transmitting and receiving an ultrasound beam to and from an examination area of a subject under examination using the ultrasound probe;
a step of generating, via at least one optical camera, an optical image by imaging the subject under examination including the ultrasound probe during examination of the examination area;
a step of storing, via a region setting memory, in the optical image divided into a plurality of regions set in advance, one region set in advance from among the plurality of regions;
a step of generating, via a trimming section, a trimmed image by trimming the one region from the optical image; and
a step of displaying, via a display control unit, the ultrasound image and the trimmed image on a monitor.

In the present invention, the ultrasound image and the trimmed image generated by trimming the region including the ultrasound probe during the examination of the examination area from the optical image are displayed on the monitor. Therefore, according to the present invention, the user can easily grasp the position and the orientation of the ultrasound probe at the time of generating the ultrasound image during the examination of the examination area of the subject under examination by viewing the trimmed image in which the ultrasound probe is enlarged.

### BRIEF DESCRIPTION OF THE DRAWINGS

FIG. 1 is a block diagram of a first embodiment showing a configuration of an ultrasound diagnostic apparatus of an embodiment of the present invention.

FIG. 2 is a block diagram of one embodiment showing a configuration of a transmission and reception circuit.

FIG. 3 is a block diagram of one embodiment showing a configuration of an ultrasound image generation unit.

FIG. 4 is a block diagram of one embodiment showing a configuration of a trimming processing unit of the ultrasound diagnostic apparatus of the first embodiment.

FIG. 5 is a flowchart of one embodiment showing an operation of the ultrasound diagnostic apparatus of the first embodiment.

FIG. 6 is a conceptual diagram of one embodiment showing a state in which a user is examining an abdomen of a subject under examination.

FIG. 7 is a conceptual diagram of one embodiment of a display screen of a monitor on which an ultrasound image and an optical image are displayed.

FIG. 8 is a conceptual diagram of one embodiment of the display screen of the monitor on which the ultrasound image and a trimmed image are displayed.

FIG. 9 is a block diagram of a second embodiment showing the configuration of the ultrasound diagnostic apparatus of the embodiment of the present invention.

FIG. 10 is a block diagram of one embodiment showing a configuration of a trimming processing unit of the ultrasound diagnostic apparatus of the second embodiment.

FIG. 11 is a flowchart of one embodiment showing an operation of the ultrasound diagnostic apparatus of the second embodiment.

FIG. 12 is a block diagram of a third embodiment showing the configuration of the ultrasound diagnostic apparatus of the embodiment of the present invention.

FIG. 13 is a block diagram of one embodiment showing a configuration of a trimming processing unit of the ultrasound diagnostic apparatus of the third embodiment.

FIG. 14 is a conceptual diagram of one embodiment of a standard optical image captured by a standard camera.

FIG. 15 is a conceptual diagram of one embodiment of a wide-angle optical image captured by a wide-angle camera.

### DESCRIPTION OF THE PREFERRED EMBODIMENTS

Hereinafter, an ultrasound diagnostic apparatus and a control method for an ultrasound diagnostic apparatus of the embodiment of the present invention will be described in detail based on suitable embodiments shown in the accompanying drawings.

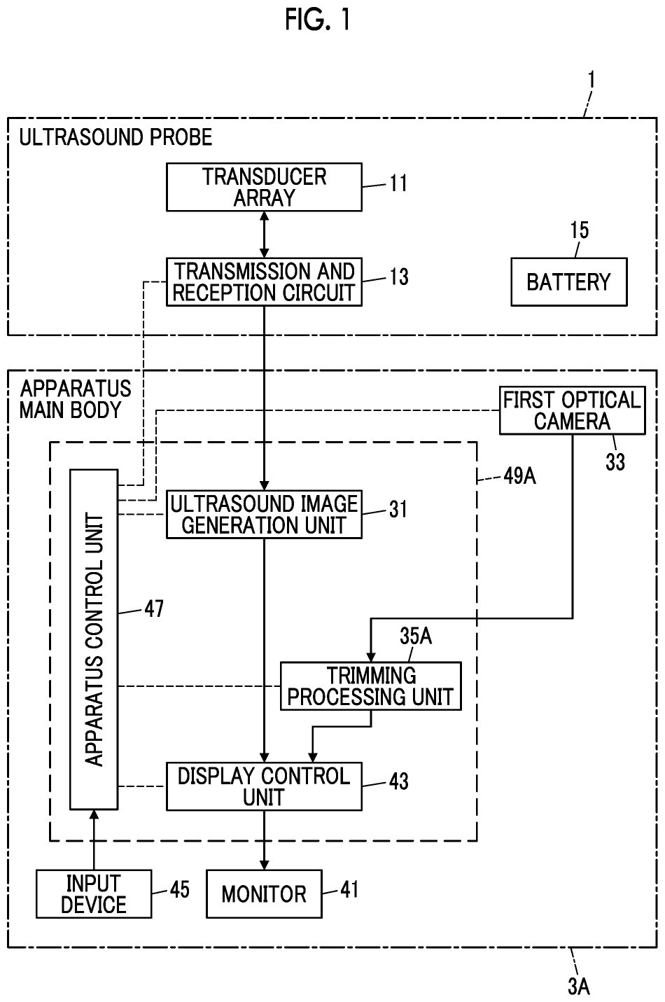

FIG. 1 is a block diagram of a first embodiment showing a configuration of the ultrasound diagnostic apparatus of the embodiment of the present invention. The ultrasound diagnostic apparatus shown in FIG. 1 is a handheld ultrasound diagnostic apparatus and comprises an ultrasound probe 1 and an apparatus main body 3A connected to the ultrasound probe 1. The ultrasound diagnostic apparatus of the present embodiment is realized by the ultrasound probe 1, the apparatus main body 3A, and an application program for ultrasound diagnosis that operates on the apparatus main body 3A.

The ultrasound probe 1 scans an examination area of a subject under examination with an ultrasound beam and outputs a sound ray signal corresponding to an ultrasound image of the examination area. As shown in FIG. 1, the ultrasound probe 1 comprises a transducer array 11, a transmission and reception circuit 13, and a battery 15. The transducer array 11 and the transmission and reception circuit 13 are bidirectionally connected, and an apparatus control unit 47 of the apparatus main body 3A, which will be described below, is connected to the transmission and reception circuit 13.

The transducer array 11 includes a plurality of ultrasound transducers arranged one-dimensionally or two-dimensionally. Each of these transducers transmits an ultrasound wave in accordance with a drive signal supplied from the transmission and reception circuit 13 and outputs an analog reception signal by receiving a reflected wave from the subject under examination.

For example, each transducer is composed of an element obtained by forming electrodes at both ends of a piezoelectric body consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), or the like.

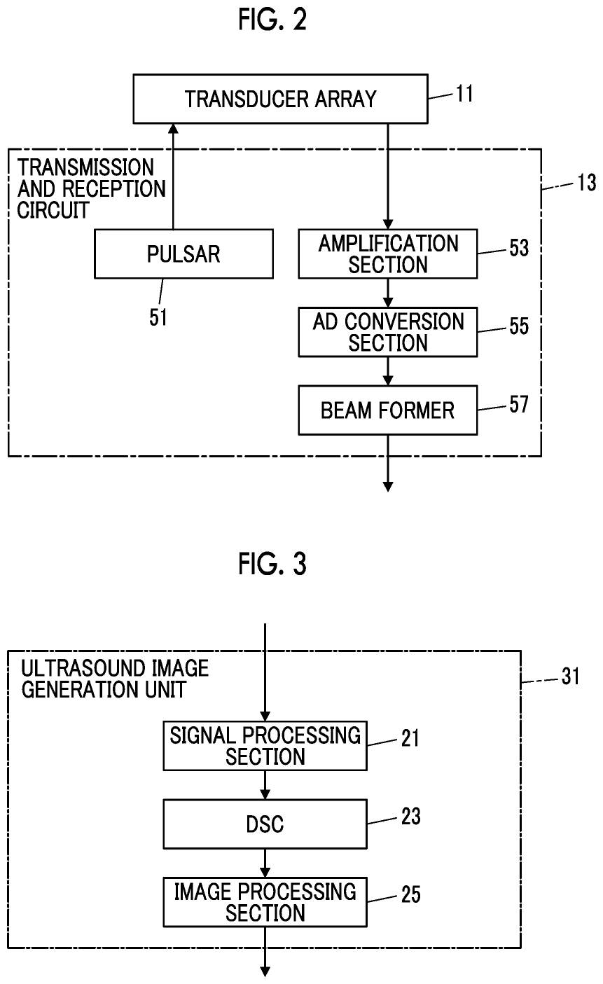

Under the control of the apparatus control unit 47, the transmission and reception circuit 13 generates the sound ray signal by transmitting an ultrasound beam from the transducer array 11 and performing reception focus processing on a reception signal output from the transducer array 11, which has received an ultrasound echo. As shown in FIG. 2, the transmission and reception circuit 13 includes a pulsar 51 connected to the transducer array 11, an amplification section 53, an analog-to-digital (AD) conversion section 55, and a beam former 57 that are sequentially connected in series to the transducer array 11.

The pulsar 51 includes, for example, a plurality of pulse generators, and performs transmission focus processing of supplying respective drive signals to the plurality of transducers by adjusting amounts of delay such that ultrasound waves transmitted from the plurality of transducers of the transducer array 11 form an ultrasound beam, based on a transmission delay pattern selected by the apparatus control unit 47. In a case where a pulsed or continuous-wave voltage is applied to the electrodes of the transducer of the transducer array 11 through the transmission focus processing, the piezoelectric body expands and contracts, and a pulsed or continuous-wave ultrasound wave is generated from each of the transducers, whereby the ultrasound beam is formed from a combined wave of these ultrasound waves.

The transmitted ultrasound beam is reflected in, for example, a target such as a site of the subject under examination and propagates toward the transducer array 11 of the ultrasound probe 1. Each of the transducers constituting the transducer array 11 expands and contracts by receiving the ultrasound echo propagating toward the transducer array 11 in this manner, generates a reception signal, which is an electrical signal, and outputs the reception signals to the amplification section 53.

The amplification section 53 amplifies the signal input from each of the transducers constituting the transducer array 11 and transmits the amplified signal to the AD conversion section 55. The AD conversion section 55 converts the analog signal transmitted from the amplification section 53 into digital reception data and outputs the reception data to the beam former 57.

The beam former 57 performs so-called reception focus processing of performing addition by applying a delay to each reception data converted by the AD conversion section 55 in accordance with a sound velocity or a sound velocity distribution set based on a reception delay pattern selected by the apparatus control unit 47. By this reception focus processing, each reception data converted by the AD conversion section 55 is phase-added, and a sound ray signal in which the focus of the ultrasound echo is narrowed down is generated.

The battery 15 is incorporated into the ultrasound probe 1 and supplies power to each circuit of the ultrasound probe 1.

Next, the apparatus main body 3A generates an ultrasound image including the examination area of the subject under examination based on the sound ray signal generated by the ultrasound probe 1 and displays the ultrasound image including the examination area of the subject under examination. The apparatus main body 3A is, for example, a handheld terminal apparatus, such as a smartphone or a tablet personal computer (PC), and comprises an ultrasound image generation unit 31, a first optical camera 33, a trimming processing unit 35A, a monitor 41, a display control unit 43, an input device 45 and an apparatus control unit 47, as shown in FIG. 1.

The ultrasound image generation unit 31 is connected to the transmission and reception circuit 13 of the ultrasound probe 1, and the display control unit 43 and the monitor 41 are sequentially connected to the ultrasound image generation unit 31. The trimming processing unit 35A and the display control unit 43 are sequentially connected to the first optical camera 33. The apparatus control unit 47 is connected to the ultrasound image generation unit 31, the first optical camera 33, the trimming processing unit 35A, and the display control unit 43, and the apparatus control unit 47 is connected to the input device 45.

The ultrasound probe 1 and the apparatus main body 3A are connected via wireless connection using wireless communication, such as wireless fidelity (Wi-Fi), or connected via wired connection using wired communication, such as a universal serial bus (USB) cable.

Under the control of the apparatus control unit 47, the ultrasound image generation unit 31 generates the ultrasound image (ultrasound image signal) including the examination area of the subject under examination from the reception signal obtained by transmitting and receiving the ultrasound beam to and from the examination area of the subject under examination using the ultrasound probe 1 (more precisely, the transducer array 11), more specifically, from the sound ray signal generated from the reception signal by the transmission and reception circuit 13. As shown in FIG. 3, the ultrasound image generation unit 31 has a configuration in which a signal processing section 21, a DSC 23, and an image processing section 25 are sequentially connected in series.

The signal processing section 21 generates image information data corresponding to the ultrasound image based on the sound ray signal generated by the transmission and reception circuit 13. More specifically, the signal processing section 21 performs signal processing on the sound ray signal generated by the beam former 57 of the transmission and reception circuit 13, for example, corrects the attenuation caused by a propagation distance according to the depth of a position where the ultrasound wave is reflected, and then performs envelope detection processing to generate the image information data representing tomographic image information regarding tissues inside the subject under examination.

The digital scan converter (DSC) 23 raster-converts the image information data generated by the signal processing section 21 into an image signal according to a scanning method of a normal television signal.

The image processing section 25 performs various types of image processing such as brightness correction, gradation correction, sharpness correction, image size correction, refresh rate correction, scanning frequency correction, and color correction according to a display format of the monitor 41 on the image signal input from the DSC 23 to generate the ultrasound image, and outputs the ultrasound image, which has been subjected to the image processing, to the display control unit 43.

The first optical camera 33 generates various optical images under the control of the apparatus control unit 47. The first optical camera 33 is disposed on a side opposite to one surface (front surface) of the apparatus main body 3A, that is, on the other surface (back surface) of the apparatus main body 3A.

The first optical camera 33 can generate the optical image including the subject under examination in a state in which the ultrasound probe 1 is in contact with the examination area, for example, by imaging the subject under examination including the ultrasound probe 1 during the examination of the examination area.

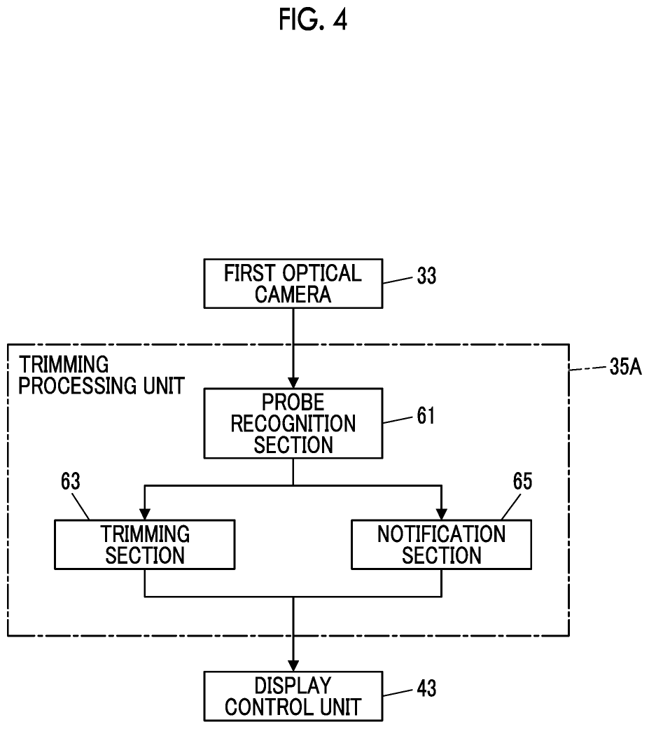

The trimming processing unit 35A performs various types of processing for generating a trimmed image from the optical image generated by the first optical camera 33 under the control of the apparatus control unit 47. As shown in FIG. 4, the trimming processing unit includes a probe recognition section 61, a trimming section 63, and a notification section 65.

The probe recognition section 61 is connected to the first optical camera 33. The trimming section 63 and the notification section 65 are each connected to the probe recognition section 61, and the display control unit 43 is connected to the trimming section 63 and the notification section 65.

The probe recognition section 61 recognizes the ultrasound probe 1 from the optical image generated by the first optical camera 33.

The probe recognition section 61 recognizes, for example, whether or not the ultrasound probe 1 is included in the optical image, in other words, whether or not the ultrasound probe 1 is shown in the optical image. In addition, the probe recognition section 61 recognizes the region of the ultrasound probe 1 in the optical image, in other words, a range in which the ultrasound probe 1 is shown in the optical image, in a case where the probe recognition section 61 recognizes that the ultrasound probe 1 is included in the optical image.

The method of recognizing the ultrasound probe 1 is not particularly limited as long as the ultrasound probe 1 can be recognized from the optical image. The probe recognition section 61 can recognize the ultrasound probe 1 from the optical image, for example, by identifying the ultrasound probe 1 included in the optical image through image recognition processing. The probe recognition section 61 can use, for example, a determination model by machine learning as image recognition processing of recognizing the ultrasound probe 1 from the optical image.

The determination model is a trained model that has learned, using a learning optical image including the ultrasound probe in a state in which the ultrasound probe is in contact with the examination area of the subject under examination as training data, a relationship between the learning optical image and the ultrasound probe included in this learning optical image for a plurality of pieces of the training data.

The determination model outputs, in response to input of an optical image as a determination target, a result of determination (prediction result) of the ultrasound probe included in this optical image based on the learning result.

The probe recognition section 61 recognizes the ultrasound probe 1 from the optical image based on the result of the determination by the determination model.

In a case where the ultrasound probe 1 is recognized from the optical image by the probe recognition section 61, the trimming section 63 generates the trimmed image by trimming a region including the ultrasound probe 1, for example, a rectangular region, from this optical image.

The trimmed image is not particularly limited as long as it is an image in which a user (examiner) of the ultrasound diagnostic apparatus can specify the position and the orientation of the ultrasound probe 1 from the trimmed image. It is desirable that the ultrasound probe 1 shown in the trimmed image is enlarged more than the ultrasound probe 1 shown in the optical image, and that the ultrasound probe 1 shown in the optical image has a size of, for example, about at least one-third and no more than half the size in the trimmed image. In addition, it is desirable that a tip portion (a contact portion with the subject under examination) of the ultrasound probe 1 is located at a center part of the trimmed image.

The notification section 65 notifies the user, in a case where the ultrasound probe 1 is not recognized from the optical image by the probe recognition section 61, in other words, until the ultrasound probe 1 is recognized from the optical image, that the ultrasound probe 1 is not recognized.

The method of the notification is not particularly limited, but for example, a message indicating that the ultrasound probe 1 is not recognized can be output from the notification section 65 to the display control unit 43 and then this message can be displayed on the monitor 41 by the display control unit 43, or this message can be output from the notification section 65 to a speaker (not shown) and then the message can be output as an audio message to be read aloud by the speaker. Alternatively, both the methods can be performed at the same time. The number of notifications is also not particularly limited, but for example, the notification may be issued only once, the notification may be issued at regular intervals, or the notification may be issued until the ultrasound probe 1 is recognized.

The display control unit 43 displays various types of information on the monitor 41 under the control of the apparatus control unit 47.

The monitor (display unit) 41 displays various types of information under the control of the display control unit 43. The monitor 41 displays, for example, the ultrasound image generated by the ultrasound image generation unit 31, and the optical image generated by the first optical camera 33 or the trimmed image thereof at the same time. The monitor 41 is disposed on the one surface of the apparatus main body 3A, that is, on a surface of the apparatus main body 3A on a side opposite to the first optical camera 33. The monitor 41 is not particularly limited, but examples thereof include a liquid crystal display (LCD) and an organic electro-luminescence (EL) display.

The input device 45 receives various instructions input from the user. The input device 45 is not particularly limited, but includes, for example, various buttons, a touch panel on which the user performs a touch operation to input various instructions, and the like.

The apparatus control unit 47 controls each unit of the ultrasound probe 1 and the apparatus main body 3A based on a program stored in advance and an instruction or the like of the user input through the input device 45.

In the case of the present embodiment, the ultrasound image generation unit 31, the trimming processing unit 35A, the display control unit 43, and the apparatus control unit 47 are configured by a processor 49A.

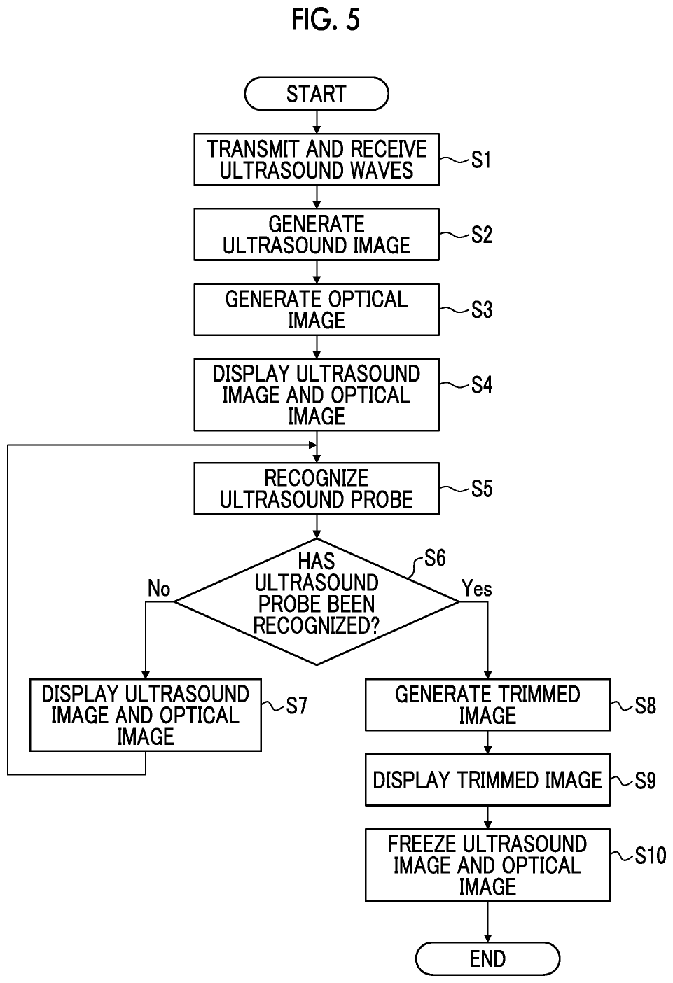

Next, the operation of the ultrasound diagnostic apparatus of the first embodiment will be described with reference to the flowchart of FIG. 5.

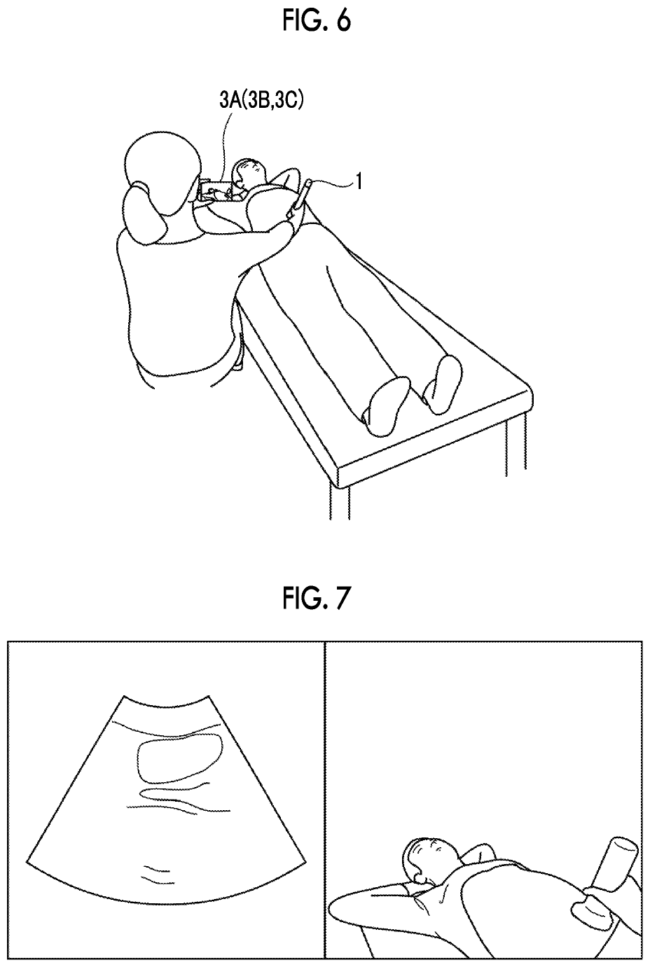

In a case of examining the subject under examination, the user first starts scanning the examination area in a state in which the ultrasound probe 1 is in contact with the examination area of the subject under examination, and images the subject under examination including the ultrasound probe 1 during the examination of the examination area by using the first optical camera 33, as shown in FIG. 6.

FIG. 6 is a conceptual diagram of one embodiment showing a state in which the user is examining an abdomen of the subject under examination. For example, in a case where the abdomen of the subject under examination is examined using the ultrasound diagnostic apparatus, the subject under examination lies supine on an examination table as shown in FIG. 6. The user holds the ultrasound probe 1 with the right hand to scans the abdomen of the subject under examination by transmitting and receiving ultrasound waves in a state in which the ultrasound probe 1 is in contact with the abdomen of the subject under examination, and at the same time, holds the apparatus main body 3A with the left hand to image the subject under examination including the ultrasound probe 1 during the examination of the abdomen by using the first optical camera 33.

In this case, under the control of the apparatus control unit 47, the transmission and reception circuit 13 performs the transmission and reception of ultrasound waves in a state in which the ultrasound probe 1 is in contact with the examination area of the subject under examination (step S1), thereby generating the sound ray signal.

That is, the ultrasound beam is transmitted to the examination area of the subject under examination from the plurality of transducers of the transducer array 11 in accordance with the drive signal from the pulsar 51.

The ultrasound echo from the examination area based on the ultrasound beam transmitted from the pulsar 51 is received by each transducer of the transducer array 11, and the reception signal, which is an analog signal, is output from each transducer of the transducer array 11 that has received the ultrasound echo.

The reception signal output from each transducer of the transducer array 11 is amplified by the amplification section 53 and is subjected to AD conversion by the AD conversion section 55, whereby the reception data is acquired.

The sound ray signal is generated by performing the reception focus processing on the reception data through the beam former 57.

Subsequently, under the control of the apparatus control unit 47, the ultrasound image generation unit 31 generates the ultrasound image including the examination area of the subject under examination based on the sound ray signal generated by the beam former 57 of the transmission and reception circuit 13 (step S2).

That is, the sound ray signal generated by the beam former 57 is subjected to various types of signal processing by the signal processing section 21, and the image information data representing tomographic image information regarding tissues inside the subject under examination is generated.

The image information data generated by the signal processing section 21 is raster-converted by the DSC 23 and is further subjected to various types of image processing by the image processing section 25, whereby the ultrasound images (video images) are sequentially generated.

Meanwhile, under the control of the apparatus control unit 47, the first optical camera 33 images the subject under examination including the ultrasound probe 1 during the examination of the examination area, and the optical images (video images) including the subject under examination in a state in which the ultrasound probe 1 is in contact with the examination area are sequentially generated (step S3).

That is, the ultrasound image including the examination area of the subject under examination and the optical image in which the subject under examination including the ultrasound probe 1 during the examination of the examination area is captured are sequentially generated at the same time.

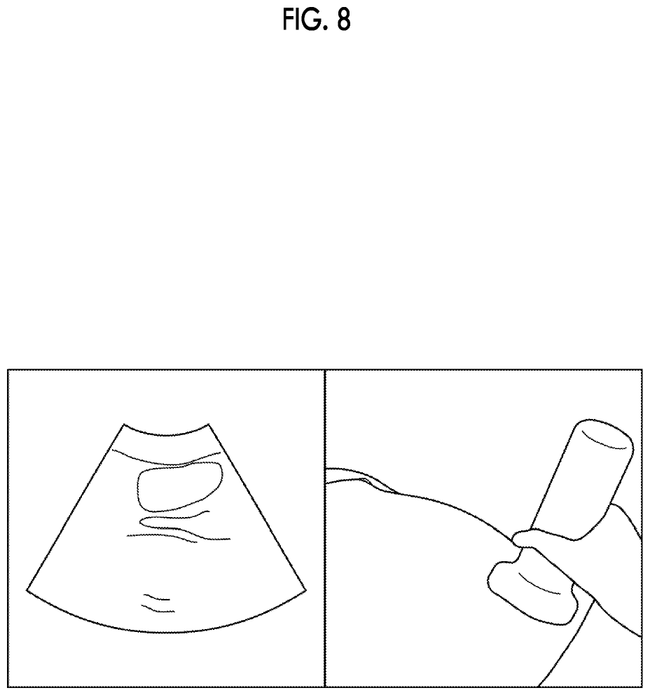

Subsequently, as shown in FIG. 7, under the control of the apparatus control unit 47, the display control unit 43 sequentially displays the ultrasound image generated by the ultrasound image generation unit 31 and the optical image generated by the first optical camera 33 on the monitor 41 (step S4).

Therefore, the user can use the ultrasound probe 1 to scan the examination area of the subject under examination to generate the ultrasound image while viewing the ultrasound image and the optical image displayed on the display screen of the monitor 41 disposed on the one surface of the apparatus main body 3A, and at the same time, can use the first optical camera 33 disposed on the other surface of the apparatus main body 3A to image the subject under examination in a state in which the ultrasound probe 1 is in contact with the examination area to generate the optical image.

Subsequently, the trimming processing unit 35A performs various types of processing for generating the trimmed image from the optical image under the control of the apparatus control unit 47.

That is, the probe recognition section 61 recognizes the ultrasound probe 1 from the optical image generated by the first optical camera 33 (step S5).

As a result, in a case where the ultrasound probe 1 is not recognized from the optical image (No in step S6), the display control unit 43 sequentially displays the ultrasound image and the optical image on the monitor 41 continuously (step S7).

In addition, in a case where the ultrasound probe 1 is not recognized, the notification section 65 notifies the user that the ultrasound probe 1 is not recognized.

After that, the process returns to step S5.

On the other hand, in a case where the ultrasound probe 1 is recognized from the optical image (Yes in step S6), the trimming section 63 generates the trimmed image by trimming a region including the ultrasound probe 1 from the optical image (step S8). For example, the size of the ultrasound probe 1 shown in the trimmed image is enlarged more than the size of the ultrasound probe 1 shown in the optical image, and the tip portion of the ultrasound probe 1 is located at the center part of the trimmed image.

In addition, the notification by the notification section 65 is stopped.

In a case where the trimmed image is generated, the display control unit 43 sequentially displays the ultrasound image and the trimmed image generated by the trimming section 63 instead of the optical image on the monitor 41 as shown in FIG. 8 (step S9).

Subsequently, the user presses a freeze button (not shown), for example, at a timing at which the user considers that a desired ultrasound image including the examination area of the subject under examination is generated, while viewing the ultrasound image and the trimmed image sequentially displayed on the monitor 41 of the apparatus main body 3A.

In a case where the freeze button is pressed, the apparatus control unit 47 freezes both the ultrasound image and the optical image (step S10).

The apparatus control unit 47 may perform control to freeze at least one of the ultrasound image or the optical image in a case where the freeze button is pressed.

Subsequently, the display control unit 43 displays, on the monitor 41, the ultrasound image (still image) and the optical image (still image) generated at the timing at which the freeze is designated by the user, and these images are stored in an image memory (not shown) in association with each other.

In the ultrasound diagnostic apparatus of the present embodiment, the ultrasound image and the trimmed image generated by trimming the region including the ultrasound probe 1 during the examination of the examination area from the optical image are displayed on the monitor 41. Therefore, the user can easily grasp the position and the orientation of the ultrasound probe 1 at the time of generating the ultrasound image during the examination of the examination area of the subject under examination by viewing the trimmed image in which the ultrasound probe 1 is enlarged.

Next, an ultrasound diagnostic apparatus according to a second embodiment will be described.

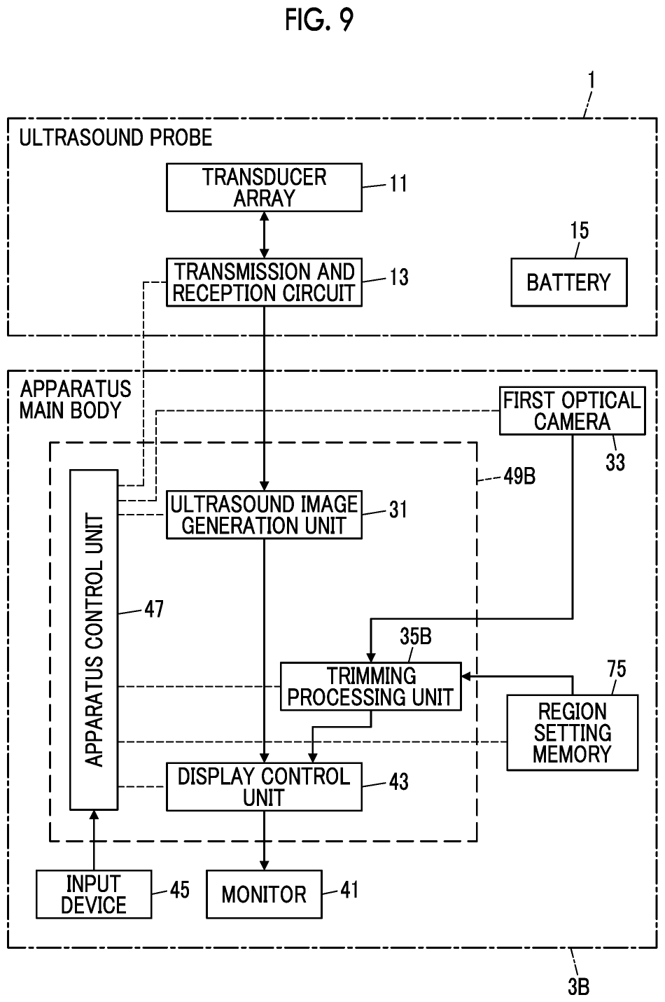

FIG. 9 is a block diagram of the second embodiment showing the configuration of the ultrasound diagnostic apparatus of the embodiment of the present invention. The ultrasound diagnostic apparatus shown in FIG. 9 comprises the ultrasound probe 1, and an apparatus main body 3B connected to the ultrasound probe 1. In addition, the apparatus main body 3B comprises the ultrasound image generation unit 31, the first optical camera 33, a trimming processing unit 35B, a region setting memory 75, the monitor 41, the display control unit 43, the input device 45, and the apparatus control unit 47.

That is, the ultrasound diagnostic apparatus of the second embodiment further comprises the region setting memory 75 with respect to the ultrasound diagnostic apparatus of the first embodiment and comprises the trimming processing unit 35B instead of the trimming processing unit 35A.

Hereinafter, the trimming processing unit 35B and the region setting memory 75 will be mainly described, and the same constituent elements as those of the ultrasound diagnostic apparatus of the first embodiment are designated by the same reference numerals, and the description thereof will not be repeated.

The trimming processing unit 35B and the display control unit 43 are sequentially connected to the first optical camera 33. The trimming processing unit 35B and the apparatus control unit 47 are connected to the region setting memory 75.

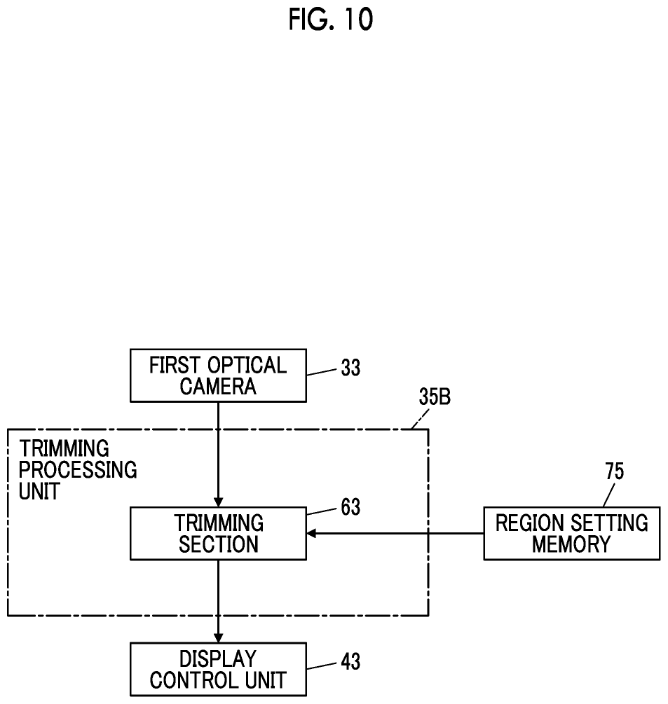

The trimming processing unit 35B performs various types of processing for generating the trimmed image from the optical image generated by the first optical camera 33 under the control of the apparatus control unit 47. As shown in FIG. 10, the trimming processing unit 35B includes the trimming section 63. The trimming section 63 is connected to the first optical camera 33 and the region setting memory 75, and the display control unit 43 is connected to the trimming section 63.

Under the control of the apparatus control unit 47, the region setting memory 75 stores, in the optical image divided into a plurality of regions set in advance, one region set in advance from among the plurality of regions. The region setting memory 75 stores, for example, in which range each of the plurality of regions set in advance is located in the optical image, and stores which region of the plurality of regions one region set in advance corresponds to.

The method of dividing the optical image is not particularly limited, but for example, in a case of a horizontally long optical image, the horizontally long optical image can be horizontally divided into two equal parts, that is, two regions: left and right, or can be divided into three equal parts, that is, three regions: left, middle, and right. Alternatively, in a case of a vertically long optical image, the vertically long optical image can be vertically divided into two equal parts, that is, two regions: top and bottom, or can be divided into three equal parts, that is, three regions: top, middle, and bottom, for example.

In the case of the present embodiment, the ultrasound image generation unit 31, the trimming processing unit 35B, the display control unit 43, and the apparatus control unit 47 are configured by a processor 49B.

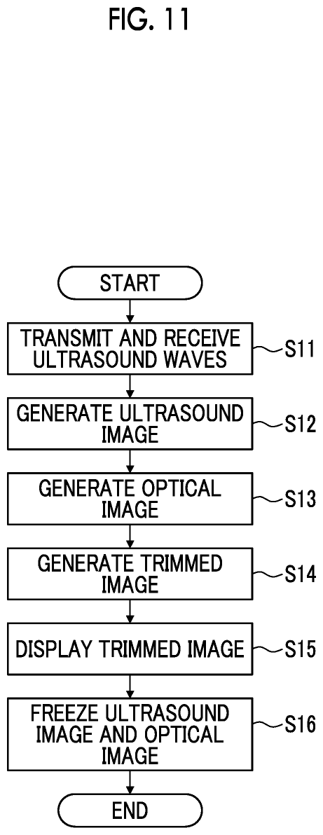

Next, the operation of the ultrasound diagnostic apparatus of the second embodiment will be described with reference to the flowchart of FIG. 11.

As shown in FIG. 6, it is assumed that, on the premise that the user holds the ultrasound probe 1 with the right hand to scan the abdomen of the subject under examination and at the same time, holds the apparatus main body 3B with the left hand to image the subject under examination including the ultrasound probe 1 during the examination of the abdomen, the region setting memory 75 stores, in the horizontally long optical image divided into three regions: left, middle, and right, as the plurality of regions set in advance, a right region as the one region set in advance from among the three regions.

The operations of the ultrasound diagnostic apparatus of the second embodiment in steps S11 to S13 are the same as the operations of the ultrasound diagnostic apparatus of the first embodiment shown in the flowchart of FIG. 5 in steps S1 to S3.

Subsequently, the trimming processing unit 35B performs various types of processing for generating the trimmed image from the optical image under the control of the apparatus control unit 47.

That is, the trimming section 63 generates the trimmed image by trimming, in the horizontally long optical image divided into three regions: left, middle, and right, stored in the region setting memory 75, the right region from among the three regions (step S14).

The subsequent operations of the ultrasound diagnostic apparatus of the second embodiment in steps S15 and S16 are the same as the operations of the ultrasound diagnostic apparatus of the first embodiment shown in the flowchart of FIG. 5 in steps S9 and S10.

As shown in FIG. 6, in a case where the user holds the ultrasound probe 1 with the right hand to scan the abdomen of the subject under examination and at the same time, holds the apparatus main body 3B with the left hand to image the subject under examination including the ultrasound probe 1 during the examination of the abdomen, there are overwhelmingly more cases where the ultrasound probe 1 is shown in the right region of the horizontally long optical image. Therefore, in this case, since the region including the ultrasound probe 1 can be trimmed by trimming the right region in the optical image, it is possible to obtain the same effect as the ultrasound diagnostic apparatus of the first embodiment even without recognizing the ultrasound probe 1 from the optical image.

In a case where the user holds the ultrasound probe 1 with the left hand to scan the abdomen of the subject under examination and at the same time, holds the apparatus main body 3B with the right hand to image the subject under examination including the ultrasound probe 1 during the examination of the abdomen, there are overwhelmingly more cases where the ultrasound probe 1 is shown in the left region of the horizontally long optical image. Therefore, in this case, by performing a setting in advance such that the left region is trimmed in the optical image, it is possible to trim the region including the ultrasound probe 1.

In addition, in a case of the vertically long optical image, there are overwhelmingly more cases where the ultrasound probe 1 is shown in a bottom region of the vertically long optical image regardless of which hand the user is holding the ultrasound probe 1 with. Therefore, in this case, by performing a setting in advance such that the bottom region is trimmed in the optical image, it is possible to trim the region including the ultrasound probe 1.

Next, an ultrasound diagnostic apparatus of a third embodiment will be described.

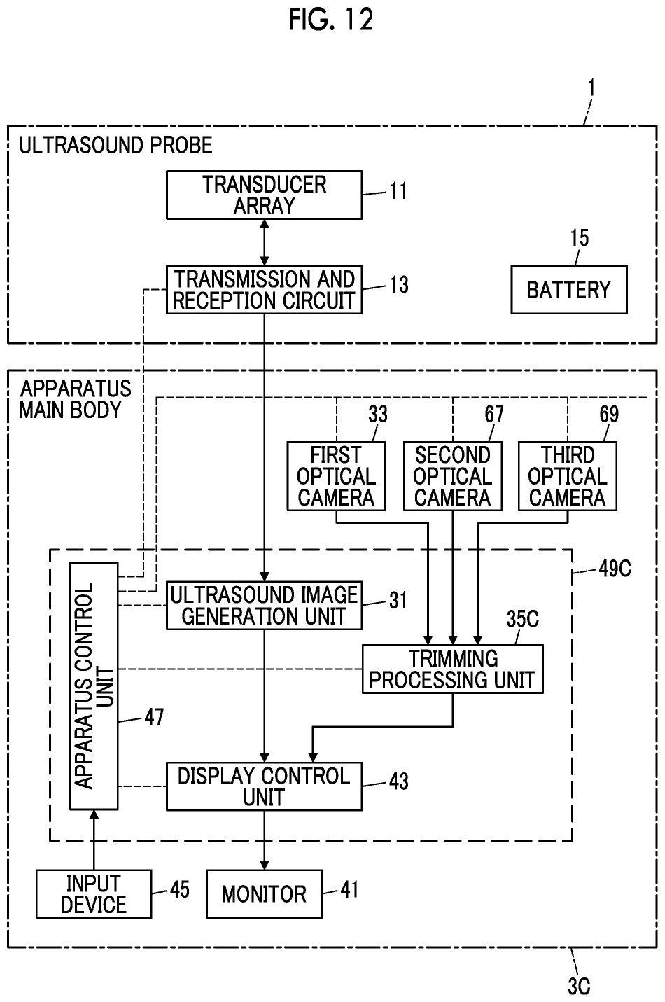

FIG. 12 is a block diagram of the third embodiment showing the configuration of the ultrasound diagnostic apparatus of the embodiment of the present invention. The ultrasound diagnostic apparatus shown in FIG. 12 comprises the ultrasound probe 1, and an apparatus main body 3C connected to the ultrasound probe 1. In addition, the apparatus main body 3C comprises the ultrasound image generation unit 31, the first optical camera 33, a second optical camera 67, a third optical camera 69, a trimming processing unit 35C, the monitor 41, the display control unit 43, the input device 45, and the apparatus control unit 47.

That is, the ultrasound diagnostic apparatus of the third embodiment further comprises the second optical camera 67 and the third optical camera 69 with respect to the ultrasound diagnostic apparatus of the first embodiment and comprises the trimming processing unit 35C instead of the trimming processing unit 35A.

Hereinafter, the second optical camera 67, the third optical camera 69, and the trimming processing unit 35C will be mainly described, and the same constituent elements as those of the ultrasound diagnostic apparatus of the first embodiment are designated by the same reference numerals, and the description thereof will not be repeated.

The trimming processing unit 35C is connected to the first optical camera 33, the second optical camera 67, and the third optical camera 69. The apparatus control unit 47 is connected to the first optical camera 33, the second optical camera 67, the third optical camera 69, and the trimming processing unit 35C.

The second optical camera 67 generates various optical images having an angle of view different from that of the first optical camera 33 under the control of the apparatus control unit 47. The second optical camera 67 is disposed on the other surface of the apparatus main body 3C. That is, the first optical camera 33 and the second optical camera 67 are disposed on the same-side surface of the apparatus main body 3C. In the same manner as the first optical camera 33, the second optical camera 67 can generate the optical image including the subject under examination in a state in which the ultrasound probe 1 is in contact with the examination area by imaging the subject under examination including the ultrasound probe 1 during the examination of the examination area.

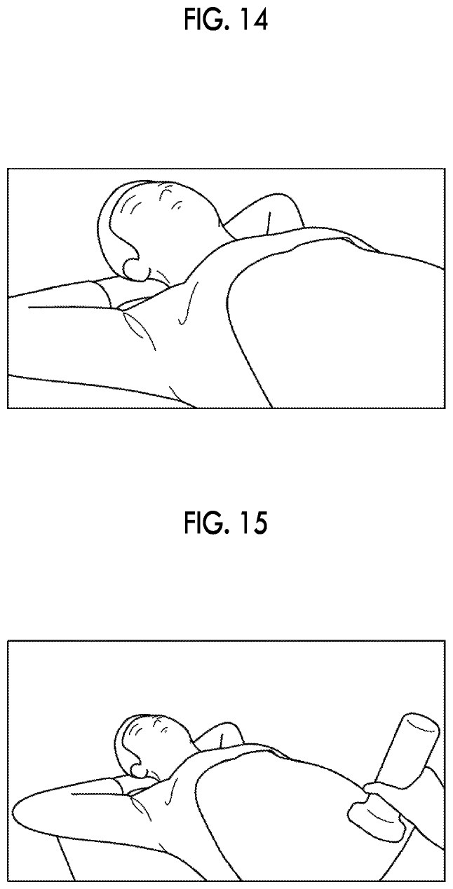

The first optical camera 33 is a standard camera that generates, for example, a standard optical image shown in FIG. 14 by imaging the subject under examination at a first angle of view, and the second optical camera 67 is a wide-angle camera that generates, for example, a wide-angle optical image shown in FIG. 15 by imaging the subject under examination at a second angle of view wider than the that of the standard camera. As described above, the apparatus main body 3C includes a plurality of optical cameras having different angles of view, including the first optical camera 33 and the second optical camera 67.

The apparatus main body 3C is not limited to the two, that is, the first optical camera 33 and the second optical camera 67, and may include two or more optical cameras having different angles of view from each other.

The third optical camera 69 generates various optical images under the control of the apparatus control unit 47. The third optical camera 69 is disposed on the one surface of the apparatus main body 3C. That is, the third optical camera 69 and the monitor 41 are disposed on the same-side surface of the apparatus main body 3C. In other words, the third optical camera 69, and the first optical camera 33 and the second optical camera 67 are disposed on surfaces of the apparatus main body 3C opposite to each other. The third optical camera 69 can generate, for example, an optical image including the user's eye by imaging the user. Further, in the same manner as the first optical camera 33, the third optical camera 69 can also generate the optical image including the subject under examination in a state in which the ultrasound probe 1 is in contact with the examination area by imaging the subject under examination including the ultrasound probe 1 during the examination of the examination area.

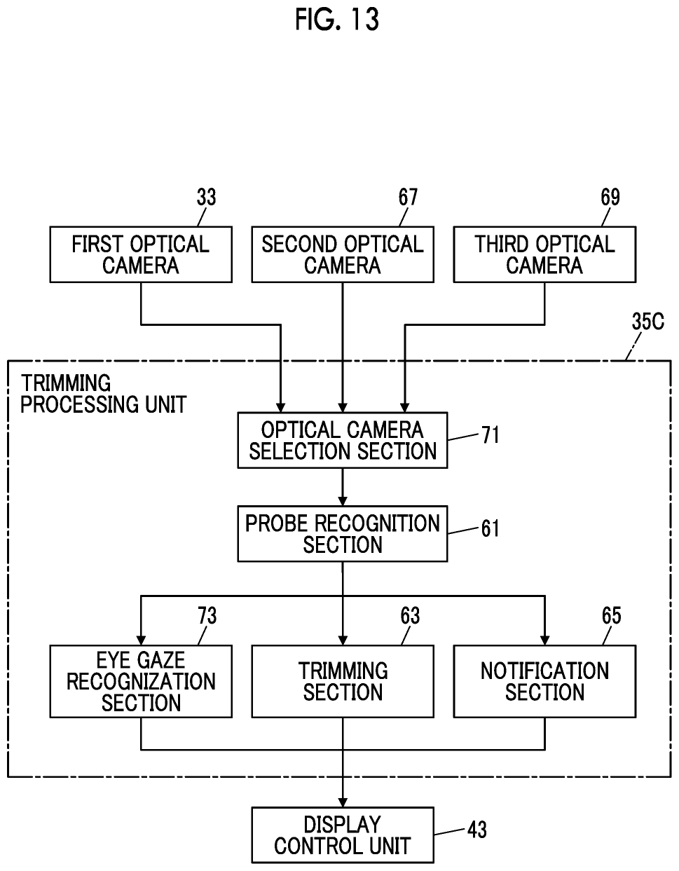

The trimming processing unit 35C performs various types of processing for generating the trimmed image from the optical image generated by any one of the first optical camera 33, the second optical camera 67, or the third optical camera 69 under the control of the apparatus control unit 47. As shown in FIG. 13, the trimming processing unit 35C includes an optical camera selection section 71, the probe recognition section 61, the trimming section 63, the notification section 65, and an eye gaze recognition section 73.

The optical camera selection section 71 is connected to the first optical camera 33, the second optical camera 67, and the third optical camera 69, and the probe recognition section 61 is connected to the optical camera selection section 71. The trimming section 63, the notification section 65, and the eye gaze recognition section 73 are each connected to the probe recognition section 61, and the display control unit 43 is connected to the trimming section 63, the notification section 65, and the eye gaze recognition section 73.

The optical camera selection section 71 selects one or more optical cameras based on an instruction from the user from among the plurality of optical cameras, including the first optical camera 33, the second optical camera 67, and the third optical camera 69, or an imaging mode corresponding to the optical camera.

The eye gaze recognition section 73 recognizes the user's eye gaze based on the optical image including the user's eye and generated by the third optical camera 69.

The eye gaze recognition section 73 recognizes whether or not an eye gaze behavior set in advance is recognized, for example, whether or not the user has winked.

The method of recognizing the eye gaze is not particularly limited as long as the user's eye gaze can be recognized from the optical image, and various conventionally known methods can be used. In addition, the eye gaze behavior set in advance is not limited to winking, and various eye gaze behaviors can be set.

In the case of the present embodiment, the ultrasound image generation unit 31, the trimming processing unit 35C, the display control unit 43, and the apparatus control unit 47 are configured by a processor 49C.

Next, the operation of the ultrasound diagnostic apparatus of the third embodiment will be described.

The user can select one or more optical cameras from among the plurality of optical cameras or an imaging mode.

In a case where the user selects one optical camera from among the plurality of optical cameras, the optical camera selection section 71 selects one optical camera from among the plurality of optical cameras based on an instruction from the user.

In cases where the first optical camera (standard camera) 33 is selected, it enters a standard imaging mode in which the standard optical image is generated by imaging the subject under examination including the ultrasound probe 1 during the examination of the examination area using the first optical camera 33.

In a case where the second optical camera (wide-angle camera) 67 is selected, it enters a wide-angle imaging mode in which the wide-angle optical image is generated by imaging the subject under examination including the ultrasound probe 1 during the examination of the examination area using the second optical camera 67.

In a case of the standard imaging mode and the wide-angle imaging mode, for example, as shown in FIG. 6, the user holds the ultrasound probe 1 with the right hand to scan the abdomen of the subject under examination and at the same time, holds the apparatus main body 3C with the left hand to image the subject under examination using the first optical camera 33.

In a case where the third optical camera 69 is selected, it enters a fixed imaging mode in which the optical image is generated by imaging the subject under examination including the ultrasound probe 1 during the examination of the examination area using the third optical camera 69.

In a case of the fixed imaging mode, for example, the user fixedly disposes the apparatus main body 3C on a far side of the subject under examination by leaning the apparatus main body 3C against a fixture, such as a stand, and in this state, the user holds the ultrasound probe 1 with the right hand to scan the abdomen of the subject under examination. In this case, the subject under examination including the ultrasound probe 1 during the examination of the examination area is imaged using the third optical camera of the apparatus main body 3C fixedly disposed.

In any case of the standard imaging mode, the wide-angle imaging mode, or the fixed imaging mode, the probe recognition section 61 recognizes the ultrasound probe 1 from the optical image generated by one optical camera selected by the optical camera selection section 71.

Then, the trimming section 63 generates the trimmed image from the optical image generated by the same one optical camera.

The first optical camera 33 and the second optical camera 67 may be disposed on the one surface (front surface) of the apparatus main body 3C, that is, on the surface of the apparatus main body 3C on the same side as the monitor 41. In this case, the subject under examination can be imaged using at least one of the first optical camera 33 or the second optical camera 67 in a state in which the apparatus main body 3C is disposed on the far side of the subject under examination by leaning the apparatus main body 3C against the fixture such as a stand. Alternatively, the plurality of optical cameras having different angles of view may be disposed on the one surface of the apparatus main body 3C, including the third optical camera 69 and a fourth optical camera (not shown). In this case, in the fixed imaging mode, it is possible to select an optical camera to be used from among the plurality of optical cameras having different angles of view.

In addition, in a case where the user selects two or more optical cameras from among the plurality of optical cameras at the same time, the optical camera selection section 71 selects the two or more optical cameras at the same time from among the plurality of optical cameras based on an instruction from the user, and it enters a simultaneous imaging mode in which the optical images are generated at the same time by imaging the subject under examination including the ultrasound probe 1 during the examination of the examination area using the selected two or more optical cameras.

For example, in a case where the first optical camera 33 and the second optical camera 67 are selected at the same time, the standard optical image is generated using the first optical camera (standard camera) 33, and at the same time, the wide-angle optical image is generated using the second optical camera (wide-angle camera) 67.

In this case, the probe recognition section 61 recognizes the ultrasound probe 1 from each of the standard optical image and the wide-angle optical image.

The probe recognition section 61 first recognizes the ultrasound probe 1 from, for example, the wide-angle optical image because the wide-angle optical image has an angle of view wider than that of the standard optical image and has a high probability of showing the ultrasound probe 1, and can recognize the ultrasound probe 1 from the standard optical image only in a case where the ultrasound probe 1 is recognized from the wide-angle optical image. Alternatively, the probe recognition section 61 can recognize the ultrasound probe 1 from each of the wide-angle optical image and the standard optical image regardless of whether or not the ultrasound probe 1 is recognized from the wide-angle optical image.

As a result, in a case where the ultrasound probe 1 is recognized from both the standard optical image and the wide-angle optical image, the trimming section 63 generates the trimmed image from the standard optical image because the standard optical image has generally higher image quality than that of the wide-angle optical image.

On the other hand, in a case where the ultrasound probe 1 is recognized from the wide-angle optical image and the ultrasound probe 1 is not recognized from the standard optical image, the trimming section 63 generates the trimmed image from the wide-angle optical image.

As described above, in the ultrasound diagnostic apparatus of the present embodiment, the user can select a desired optical camera from among the plurality of optical cameras, and the optical image can be generated using the optical camera selected by the user.

Meanwhile, in a case where the user holds the ultrasound probe 1 with the right hand and the apparatus main body 3C with the left hand, it may be difficult to press the freeze button. In response to this, in the ultrasound diagnostic apparatus of the present embodiment, a freeze instruction can be issued using the user's eye gaze.

In this case, the user is imaged by the third optical camera 69 disposed on the surface of the apparatus main body 3C on the same side as the monitor 41, and the optical image including the user's eye is generated.

Subsequently, the eye gaze recognition section 73 recognizes the user's eye gaze based on the optical image generated by the third optical camera 69.

Then, in a case where the eye gaze behavior set in advance by the eye gaze recognition section 73, for example, the winking of the user is recognized, the apparatus control unit 47 freezes both the ultrasound image and the optical image.

Alternatively, the freeze instruction may be issued using the voice of the user.

In this case, in a case where the user utters a keyword set in advance, a voice recognition section (not shown) recognizes the keyword uttered by the user.

Subsequently, in a case where a keyword set in advance, for example, a voice of “freeze” is recognized by the voice recognition section, the apparatus control unit 47 freezes both the ultrasound image and the optical image.

In this way, the user can easily issue the freeze instruction using the eye gaze or the voice.

As described above, the apparatus control unit 47 may perform control to freeze both the ultrasound image and the optical image at the same time, but perform control to freeze at least one of the ultrasound image or the optical image.

Although the ultrasound diagnostic apparatuses of the first embodiment, the second embodiment, and the third embodiment have been described, these embodiments may also be implemented alone or in combination.

For example, in the apparatus main body 3A of the ultrasound diagnostic apparatus of the first embodiment, the region setting memory 75 of the ultrasound diagnostic apparatus of the second embodiment may be provided to divide the optical image into a plurality of regions. Further, in this case, the probe recognition section 61 may recognize the ultrasound probe 1 from only one region among the plurality of regions, and the trimming section 63 may generate the trimmed image from only the same one region.

In addition, the apparatus main bodies 3A and 3B of the ultrasound diagnostic apparatuses of the first embodiment and the second embodiment are each not limited to the first optical camera 33 and need only each comprise at least one optical camera. For example, in the apparatus main bodies 3A and 3B, a plurality of optical cameras having different angles of view may be provided according to the third embodiment, and one or more optical cameras may be selected from among the plurality of optical cameras based on an instruction from the user. In this case, the probe recognition section 61 recognizes the ultrasound probe 1 from the optical image generated by at least one optical camera, and the trimming section 63 trims a region including the ultrasound probe from one optical image in which the ultrasound probe 1 is recognized by the probe recognition section 61, among the optical images generated by the at least one optical camera.

In addition, the user may use the input device 45 to set a trimmed region in the optical image displayed on the monitor 41.

In this case, the probe recognition section 61 recognizes the ultrasound probe 1 from only the trimmed region set based on an instruction from the user in the optical image.

Then, the trimming section 63 generates the trimmed image from only this trimmed region.

In the ultrasound diagnostic apparatus, a plurality of the ultrasound probes may be switched and used. Further, it is desirable that the probe recognition section 61 recognizes the ultrasound probe 1 in a state of being held by the user's hand from the optical image. By recognizing the ultrasound probe 1 in a state of being held by the user's hand, it is possible to correctly recognize the ultrasound probe 1 during the examination of the examination area without erroneous recognition even in a case where the plurality of ultrasound probes are shown in the optical image.

In addition, in a case where only the ultrasound probe 1 in a state of being held by the user's hand is zoomed in, it may be difficult to discern which site of the subject under examination the examination area corresponds to. Therefore, it is desirable that the trimming section 63 generates the trimmed image by trimming from the optical image, for example, a rectangular region including the ultrasound probe 1 and the subject under examination in a range representing the position and the orientation of the ultrasound probe 1 during the examination of the examination area, in other words, the subject under examination in a range in which the user can specify the position and the orientation of the ultrasound probe 1.

The apparatus main bodies 3A, 3B, and 3C may comprise the ultrasound image generation unit 31, but the present invention is not limited to this, and all or only the signal processing section 21 of the ultrasound image generation unit 31 may be provided on an ultrasound probe 1 side.

In the apparatus of the embodiment of the present invention, as the hardware configuration of the processing unit that executes various types of processing, such as the transmission and reception circuit 13, the ultrasound image generation unit 31, the trimming processing units 35A, 35B, and 35C, the display control unit 43, and the apparatus control unit 47, dedicated hardware may be used, or various processors or computers that execute programs may be used. In addition, as the image memory, a semiconductor memory may be used, recording media, such as a flash memory, a secure digital card (SD card), or a universal serial bus memory (USB memory), may be used, or a hard disk drive (HDD), a solid state drive (SSD), an external server, or the like can also be used.

The various processors include a central processing unit (CPU) which is a general-purpose processor that executes software (programs) to function as various processing units, a programmable logic device (PLD) which is a processor whose circuit configuration is changeable after manufacturing, such as a field programmable gate array (FPGA), and a dedicated electric circuit which is a processor that has a dedicated circuit configuration designed to perform specific processing, such as an application specific integrated circuit (ASIC), and the like.

One processing unit may be composed of one of these various processors or may be composed of a combination of two or more processors of the same type or different types, for example, a combination of a plurality of FPGAs, a combination of an FPGA and a CPU, or the like. In addition, a plurality of processing units may be composed of one of the various processors, or two or more of the plurality of processing units may be collectively composed of one processor.

For example, there is an aspect in which one or more CPUs and software are combined to constitute one processor and the processor functions as a plurality of processing units, as represented by a computer such as a client and a server. In addition, there is an aspect in which a processor that realizes functions of an entire system including a plurality of processing units with one integrated circuit (IC) chip is used, as represented by a system on chip (SoC) or the like.

Further, as the hardware configuration of these various processors, more specifically, an electric circuit (circuitry) in which circuit elements, such as semiconductor elements, are combined is used.

In addition, the method of the embodiment of the present invention can be implemented, for example, by a program for causing a computer to execute each of the steps. Further, it is also possible to provide a computer-readable recording medium on which the program is recorded.

Although the present invention has been described in detail above, the present invention is not limited to the above-described embodiment, and various modifications or changes may be made without departing from the gist of the present invention, of course.

### EXPLANATION OF REFERENCES

1: ultrasound probe
3A, 3B, 3C: apparatus main body
11: transducer array
13: transmission and reception circuit
15: battery
21: signal processing section
23: DSC
25: image processing section
31: ultrasound image generation unit
33: first optical camera
35B, 35C: trimming processing unit
41: monitor
43: display control unit
45: input device
47: apparatus control unit
49A, 49B, 49C: processor
51: pulsar
53: amplification section
55: AD conversion section
57: beam former
61: probe recognition section
63: trimming section
65: notification section
67: second optical camera
69: third optical camera
71: optical camera selection section
73: eye gaze recognition section
75: region setting memory

What is claimed is:

1. An ultrasound diagnostic apparatus comprising:
an ultrasound probe; and
an apparatus main body connected to the ultrasound probe,
wherein the apparatus main body includes
an ultrasound image generation unit configured to generate an ultrasound image from a reception signal obtained by transmitting and receiving an ultrasound beam to and from an examination area of a subject under examination using the ultrasound probe,
at least one optical camera configured to generate an optical image by imaging the subject under examination including the ultrasound probe during examination of the examination area,
a probe recognition section configured to recognize the ultrasound probe from the optical image,
a trimming section configured to generate a trimmed image by trimming a region including the ultrasound probe from the optical image in a case where the ultrasound probe is recognized,
a monitor, and
a display control unit configured to display the ultrasound image and the trimmed image on the monitor,
the probe recognition section is configured to recognize the ultrasound probe from only a specific region of the optical image, and
the trimming section is configured to generate the trimmed image from only the specific region.

2. The ultrasound diagnostic apparatus according to claim 1,
wherein the at least one optical camera includes a plurality of optical cameras having different angles of view from each other,
the apparatus main body includes an optical camera selection section configured to select one or more optical cameras from among the plurality of optical cameras based on an instruction from a user, and
the plurality of optical cameras are disposed on the same-side surface of the apparatus main body.

3. The ultrasound diagnostic apparatus according to claim 2,
wherein the optical camera selection section is configured to select one optical camera from among the plurality of optical cameras based on an instruction from the user,
the probe recognition section is configured to recognize the ultrasound probe from an optical image generated by the one optical camera, and
the trimming section is configured to generate the trimmed image from the optical image generated by the one optical camera.

4. The ultrasound diagnostic apparatus according to claim 2,
wherein the optical camera selection section is configured to select a first optical camera and a second optical camera from among the plurality of optical cameras based on an instruction from the user,
the first optical camera is a standard camera that generates a standard optical image by imaging the subject under examination with a first angle of view,
the second optical camera is a wide-angle camera that generates a wide-angle optical image by imaging the subject under examination with a second angle of view having a wider angle of view than that of the standard camera, and
the probe recognition section is configured to recognize the ultrasound probe from each of the standard optical image and the wide-angle optical image.

5. The ultrasound diagnostic apparatus according to claim 4,
wherein the trimming section is configured to generate the trimmed image from the standard optical image in a case where the ultrasound probe is recognized from both the standard optical image and the wide-angle optical image, and to generate the trimmed image from the wide-angle optical image in a case where the ultrasound probe is recognized from the wide-angle optical image and the ultrasound probe is not recognized from the standard optical image.

6. The ultrasound diagnostic apparatus according to claim 4,
wherein the probe recognition section is configured to recognize the ultrasound probe from the wide-angle optical image, and to recognize the ultrasound probe from the standard optical image in a case where the ultrasound probe is recognized from the wide-angle optical image.

7. The ultrasound diagnostic apparatus according to claim 1,
wherein the apparatus main body includes a region setting memory configured to store, in the optical image divided into a plurality of regions set in advance, one region set in advance from among the plurality of regions,
the probe recognition section is configured to recognize the ultrasound probe from only the one region, and
the trimming section is configured to generate the trimmed image from only the one region.

8. The ultrasound diagnostic apparatus according to claim 1,
wherein the probe recognition section is configured to recognize, in the optical image, the ultrasound probe from only a trimmed region set based on an instruction from a user, and
the trimming section is configured to generate the trimmed image from only the trimmed region.

9. The ultrasound diagnostic apparatus according to claim 1,
wherein the probe recognition section is configured to recognize the ultrasound probe in a state of being held by a user's hand from the optical image.

10. The ultrasound diagnostic apparatus according to claim 1,
wherein the apparatus main body includes a notification section configured to issue a notification that the ultrasound probe is not recognized in a case where the ultrasound probe is not recognized.

11. The ultrasound diagnostic apparatus according to claim 10,
wherein the display control unit is configured to display the optical image on the monitor in a case where the ultrasound probe is not recognized, and to display the trimmed image on the monitor in a case where the ultrasound probe is recognized.

12. An ultrasound diagnostic apparatus comprising:
an ultrasound probe; and
an apparatus main body connected to the ultrasound probe,
wherein the apparatus main body includes
an ultrasound image generation unit configured to generate an ultrasound image from a reception signal obtained by transmitting and receiving an ultrasound beam to and from an examination area of a subject under examination using the ultrasound probe,
at least one optical camera configured to generate an optical image by imaging the subject under examination including the ultrasound probe during examination of the examination area,
a region setting memory configured to store, in the optical image divided into a plurality of regions set in advance, one region set in advance from among the plurality of regions,
a trimming section configured to generate a trimmed image by trimming the one region from the optical image,
a monitor, and
a display control unit configured to display the ultrasound image and the trimmed image on the monitor,
the monitor is disposed on one surface of the apparatus main body, and the at least one optical camera includes a plurality of optical cameras disposed on another surface of the apparatus main body,
a wide-angle camera of the plurality of optical cameras is used to generate a wide-angle optical image, and
the trimming section is configured to generate the trimmed image from the wide-angle optical image.

13. The ultrasound diagnostic apparatus according to claim 12,
wherein the at least one optical camera includes a plurality of optical cameras having different angles of view from each other,
the apparatus main body includes an optical camera selection section configured to select one or more optical cameras from among the plurality of optical cameras based on an instruction from a user,
the plurality of optical cameras are disposed on the same-side surface of the apparatus main body,
the optical camera selection section is configured to select one optical camera from among the plurality of optical cameras based on an instruction from the user, and
the trimming section is configured to generate the trimmed image from an optical image generated by the one optical camera.

14. The ultrasound diagnostic apparatus according to claim 1,
wherein the at least one optical camera includes a third optical camera disposed on a surface of the apparatus main body on the same side as the monitor, and
the apparatus main body includes
an eye gaze recognition section configured to recognize a user's eye gaze based on an optical image generated by the third optical camera, and
an apparatus control unit configured to freeze at least one of the ultrasound image or the optical image in a case where an eye gaze behavior set in advance is recognized.

15. The ultrasound diagnostic apparatus according to claim 1,
wherein the trimming section is configured to generate the trimmed image by trimming from the optical image, a region including, in addition to the ultrasound probe, the subject under examination in a range representing a position and an orientation of the ultrasound probe during the examination of the examination area.

16. A control method for an ultrasound diagnostic apparatus, comprising:
a step of generating, via an ultrasound image generation unit, an ultrasound image from a reception signal obtained by transmitting and receiving an ultrasound beam to and from an examination area of a subject under examination using the ultrasound probe;
a step of generating, via at least one optical camera, an optical image by imaging the subject under examination including the ultrasound probe during examination of the examination area;
a step of recognizing, via a probe recognition section, the ultrasound probe from the optical image;
a step of generating, via a trimming section, a trimmed image by trimming a region including the ultrasound probe from the optical image in a case where the ultrasound probe is recognized; and
a step of displaying, via a display control unit, the ultrasound image and the trimmed image on a monitor,
wherein the step of recognizing the ultrasound probe recognizes the ultrasound probe from only a specific region of the optical image, and
the step of generating the trimmed image generates the trimmed image from only the specific region.

17. A control method for an ultrasound diagnostic apparatus, comprising:
a step of generating, via an ultrasound image generation unit, an ultrasound image from a reception signal obtained by transmitting and receiving an ultrasound beam to and from an examination area of a subject under examination using the ultrasound probe;
a step of generating, via at least one optical camera, an optical image by imaging the subject under examination including the ultrasound probe during examination of the examination area;
a step of storing, via a region setting memory, in the optical image divided into a plurality of regions set in advance, one region set in advance from among the plurality of regions;
a step of generating, via a trimming section, a trimmed image by trimming the one region from the optical image; and
a step of displaying, via a display control unit, the ultrasound image and the trimmed image on a monitor,
wherein the monitor is disposed on one surface of the apparatus main body, and the at least one optical camera includes a plurality of optical cameras disposed on another surface of the apparatus main body,
the step of generating the optical image uses a wide-angle camera of the plurality of optical cameras to generate a wide-angle optical image, and
the step of generating the trimmed image generates the trimmed image from the wide-angle optical image.

18. The ultrasound diagnostic apparatus according to claim 5,
wherein the probe recognition section is configured to recognize the ultrasound probe from the wide-angle optical image, and to recognize the ultrasound probe from the standard optical image in a case where the ultrasound probe is recognized from the wide-angle optical image.

19. The ultrasound diagnostic apparatus according to claim 2,
wherein the apparatus main body includes a region setting memory configured to store, in the optical image divided into a plurality of regions set in advance, one region set in advance from among the plurality of regions,
the probe recognition section is configured to recognize the ultrasound probe from only the one region, and
the trimming section is configured to generate the trimmed image from only the one region.

20. The ultrasound diagnostic apparatus according to claim 2,
wherein the probe recognition section is configured to recognize, in the optical image, the ultrasound probe from only a trimmed region set based on an instruction from a user, and
the trimming section is configured to generate the trimmed image from only the trimmed region.
